(19) United States
(12) Patent Application Publication
(10) Pub. No.: US 2023/0210998 A1
(43) Pub. Date: Jul. 6, 2023

(54) METHODS FOR THE CONJUGATION OF ANTHRACYCLINES TO CARBOHYDRATE POLYMERIC CARRIERS

(71) Applicant: Navidea Biopharmaceuticals, Inc., Dublin, OH (US)

(72) Inventors: David A. Ralph, Columbus, OH (US); Jeffrey Arnold, Andover, MA (US)

(21) Appl. No.: 18/148,023

(22) Filed: Dec. 29, 2022

Related U.S. Application Data
(60) Provisional application No. 63/294,996, filed on Dec. 30, 2021.

(51) Int. Cl. A61K 47/61; C07D 309/02; C07D 235/04

(52) U.S. Cl. CPC A61K 47/61; C07D 309/02; C07D 235/04

(57) ABSTRACT

Methods, compounds, and compositions of conjugating anthracyclines to a carbohydrate polymer backbone via click chemistry are provided. The conjugation of anthracyclines utilizing a reaction between a hydrazone azide moiety and alkyne moiety provide for compositions with less crosslinking, and thereby increasing the efficacy of targeted drug delivery. The methods further provide for controlled loading of anthracycline to a carbohydrate polymer backbone.

### CROSS-REFERENCE TO RELATED APPLICATIONS

This application claims the benefit of U.S. Provisional Application Ser. No. 63/294,996, filed on Dec. 30, 2021 and entitled “Methods for the Conjugation of Anthracyclines to Polymeric Carriers,” the entire contents of which are incorporated herein by reference.

### TECHNICAL FIELD

The disclosure relates to the synthesis of chemical compounds, methods of making, and methods of use thereof for the targeted delivery of therapeutic drugs, including compounds wherein doxorubicin and/or anthracyclines are added to mannosylated amine dextrans (MAD) and/or other carbohydrate polymeric backbones.

### BACKGROUND

Targeted drug delivery can provide a number of benefits including allowing relatively higher and potentially more efficacious drug concentrations at sites of disease, while avoiding potentially toxic or unwanted side effects in pathologically uninvolved tissues. Targeted drug delivery involves associating or combining a drug of interest/payload with a vehicle that can hold or sequester the drug payload, preventing interaction with off-target tissues. The drug delivery vehicle should also possess the ability to associate itself with intended pathological lesions and subsequently release its drug payload. This combination of features—targeted delivery and selective release—can be challenging to realize in a drug delivery vehicle/drug payload molecular construct.

Current constructs attaching a therapeutic agent, such as doxorubicin or other anthracyclines, to a MAD polymer have faced challenges in providing reliable and scalable results. For example, current methods for attaching doxorubicin to a free amine of a MAD backbone are deficient in that they result in doxorubicin conjugated products that have grossly crosslinked MAD backbones with undesirable insolubility and high molecular weight distributions, which adversely affect biodistribution and the ability to penetrate into target tissues. With current methods of attaching doxorubicin to a MAD backbone, an acid-catalyzed hydrazone conjugation of doxorubicin to a MAD may be utilized, where doxorubicin is added under highly acidic conditions to create the final conjugate structure. The current methods result in products with undesirably low numbers of conjugated doxorubicin moieties.

The deficiencies associated with the current methods of attaching doxorubicin to MAD backbones result in compositions with poor batch to batch reproducibility, and as a result, difficulties in predictably altering the loading ratios of doxorubicin.

There is a need in the art for improved compositions and synthesis methods for targeted drug delivery.

### SUMMARY

Disclosed herein are various compositions for targeted drug delivery. Further disclosed herein are methods for synthesis of compositions for targeted drug delivery.

In Example 1, a method for conjugating anthracycline to a carbohydrate polymer comprises providing (1) a carbohydrate polymer comprising at least one alkyne moiety bound directly or indirectly thereto, and (2) an anthracycline derivatized to a hydrazone azide; or providing (1) a carbohydrate polymer comprising at least one hydrazone azide moiety bound directly or indirectly thereto, and (2) an anthracycline comprising at least one alkyne moiety bound directly or indirectly thereto; and reacting the carbohydrate polymer with the anthracycline in the presence of a Cu¹ catalyst and an amine ligand to form a 1,3-triazole linkage between the anthracycline and the carbohydrate polymer. In embodiments, the 1,3-triazole linkage results from a 1,3-cycloaddition between the azide and the alkyne.

Example 2 relates to the method according to Example 1, wherein the carbohydrate polymer is selected from the group consisting of cellulose, dextran, and mannan.

Example 3 relates to the method according to Example 1 or Example 2, wherein the anthracycline comprises doxorubicin.

In Example 4, a method of synthesizing an anthracycline carbohydrate polymer construct comprises providing an alkyne carbohydrate polymer, providing an anthracycline hydrazone azide, and reacting the alkyne carbohydrate polymer with the anthracycline hydrazone azide in the presence of a Cu¹ catalyst and a ligand to form a 1,3-triazole linkage between the anthracycline and carbohydrate polymer. In embodiments, the 1,3-triazole linkage results from a 1,3-cycloaddition between the azide of the anthracycline hydrazone azide and the alkyne of the alkyne carbohydrate polymer.

Example 5 relates to the method according to Example 4, wherein the anthracycline hydrazone azide is formed by a step comprising derivatizing the anthracycline by reacting the anthracycline with a hydrazide via condensation to form the anthracycline hydrazone azide.

Example 6 relates to the method according to Example 5, wherein the hydrazide is 4-azidobenzohydrazide.

Example 7 relates to the method according to any one of Examples 4 to Example 6, wherein the anthracycline comprises doxorubicin.

Example 8 relates to the method according to any one of Examples 4 to Example 7, wherein the alkyne carbohydrate polymer construct is synthesized by a step comprising reacting an amine carbohydrate polymer comprising cellulose, dextran, or mannan, with an alkyne under dehydrative conditions to form the alkyne carbohydrate polymer.

Example 9 relates to the method according to Example 8, wherein the alkyne is hexynoic acid.

Example 10 relates to the method according to any one of Example 4 to Example 9, wherein the ligan is an amine ligand.

Example 11 relates to the method according to Example 10, wherein the amine ligand has the following structure:

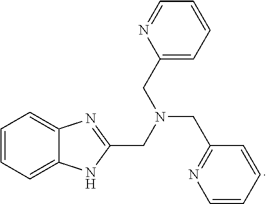 [SMILES: c1ccc(CN(Cc2ccccn2)Cc2nc3ccccc3[nH]2)nc1]

Example 12 relates to the method according to Example 11, wherein the ligand is synthesized through the reaction of ((2-benzimidazolyl)methyl)amine and 2-(chloromethyl)pyridine in the presence of Et₃N.

Example 13 relates to the method according to any one of Examples 4 to 12, wherein the ligand and Cu¹ catalyst are present at a ratio of between about 1:5 to about 5:1.

Example 14 relates to the method according to any one of Examples 4 to 13, wherein the step of reacting the alkyne carbohydrate polymer with the anthracycline hydrazone azide is carried out in the presence of sodium ascorbate.

Example 15 relates to the method according to Example 14, wherein the Cu¹ catalyst and ascorbate are present at a ratio of between about 1:5 to about 9:10.

Example 16 relates to the method according to any one of Examples 4 to 15, wherein the alkyne and the Cu¹ catalyst are present at a ratio of between about 1:1 to about 5:1.

Example 17 relates to the method according to any one of Examples 4 to 16, wherein the reacting step is performed in the presence of DMSO and water, and wherein the DMSO and water are present at a ratio of between about 4:1 to about 20:1.

Example 18 relates to the method according to any one of Examples 4 to 17, wherein loading of anthracycline to the anthracycline carbohydrate polymer construct can be controlled by varying the ratio of alkyne to the anthracycline hydrazone azide.

Example 19 relates to the method according to Example 18, wherein as the ratio of the alkyne to anthracycline hydrazone azide increases, the loading of anthracycline to the anthracycline carbohydrate polymer construct decreases.

Example 20 relates to the method according to any one of Examples 4 to 19, wherein the alkyne carbohydrate polymer is a mannosylated alkyne carbohydrate polymer having a molecular weight (Mw) of from about 1 kDa to about 50 kDa.

Example 21 relates to the method according to any one of Examples 4 to 20, wherein the alkyne carbohydrate polymer has the structure of formula (I):

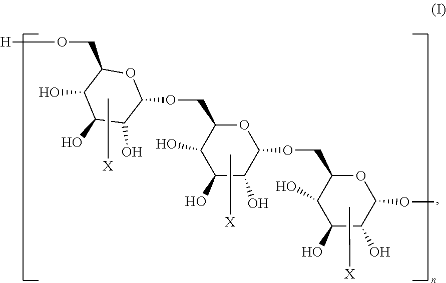

wherein each X is independently H, L2-R, or L3-Y; each L2 and L3 are independently amino terminated leashes comprising the formula —(CH₂)ₚS(CH₂)_{q}—NH—, wherein p and q are integers from 0 to 5; each R independently comprises a mannose-binding C-type lectin receptor targeting moiety, or H; each Y independently comprises a terminal alkyne moiety, or H; and n is an integer greater than zero, wherein at least one R comprises a mannose-binding C-type lectin receptor targeting moiety selected from the group consisting of mannose, fucose, and n-acetylglucosamine; wherein at least one Y comprises the terminal alkyne moiety; and wherein each unit of n may be the same or different.

Example 22 relates to the method according to Example 21, wherein each X is independently H

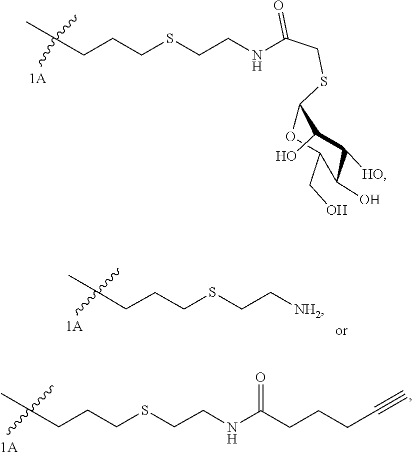

and wherein each bond 1A is connected to any —OH group in formula (I).

Example 23 relates to the method according to any one of Examples 4 to 22, wherein the reacting step is carried out at a pH of from about 6.5 to about 10.

Example 24 relates to the method according to any one of Examples 4 to 23, wherein the anthracycline is released from the anthracycline carbohydrate polymer construct at a pH of about 5.5 or below.

Example 25 relates to the method according to any one of Examples 4 to 24, wherein the anthracycline carbohydrate polymer has the structure of formula (II):

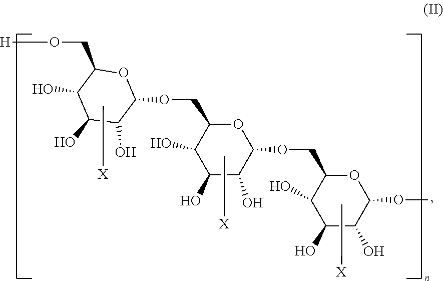

wherein each X is independently H, L1-A, L2-R, or L3-Y; each L1, L2 and L3 are independently amino terminated leashes comprising the formula —(CH₂)ₚS(CH₂)_{q}—NH—, wherein p and q are integers from 0 to 5; each A independently comprises an anthracycline hydrazone azide moiety, or H; each R independently comprises a mannose-binding C-type lectin receptor targeting moiety, or H; each Y independently comprises a terminal alkyne moiety, or H; and n is an integer greater than zero, wherein at least one A comprises the anthracycline hydrazone azide moiety; wherein at least one R comprises a mannose-binding C-type lectin receptor targeting moiety selected from the group consisting of mannose, fucose, and n-acetylglucosamine; wherein at least one Y comprises the terminal alkyne moiety; and wherein each unit of n may be the same or different.

Example 26 is related to the method according to Example 25, wherein each X is independently H,

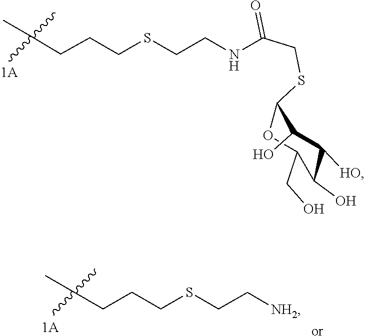

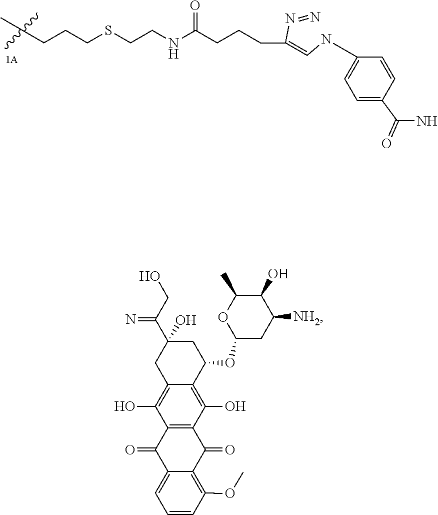

and wherein each bond 1A is connected to any —OH group in formula (II).

In Example 27, a pharmaceutical composition comprises a compound having the structure of formula (II):

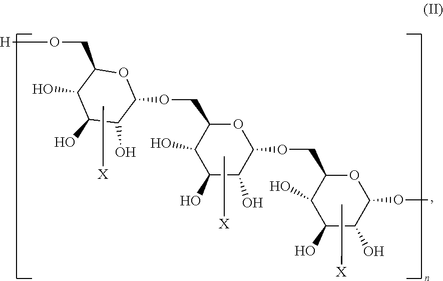

wherein each X is independently H, L1-A, L2-R, or L3-Y; each L1, L2 and L3 are independently amino terminated leashes comprising the formula —(CH₂)ₚS(CH₂)_{q}—NH—, wherein p and q are integers from 0 to 5; each A independently comprises an anthracycline hydrazone azide moiety, or H; each R independently comprises a mannose-binding C-type lectin receptor targeting moiety, or H; each Y independently comprises a terminal alkyne moiety, or H; and n is an integer greater than zero, wherein at least one A comprises the anthracycline hydrazone azide moiety; wherein at least one R comprises a mannose-binding C-type lectin receptor targeting moiety selected from the group consisting of mannose, fucose, and n-acetylglucosamine; wherein at least one Y comprises the terminal alkyne moiety; wherein each unit of n may be the same or different; and a pharmaceutically acceptable carrier thereof.

Example 28 relates to the pharmaceutical composition of Example 27, wherein each X is independently H,

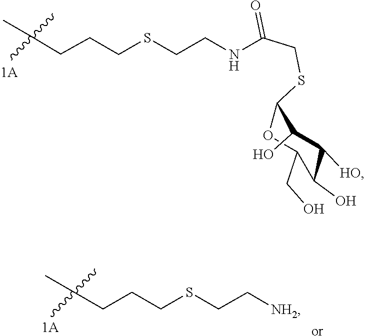

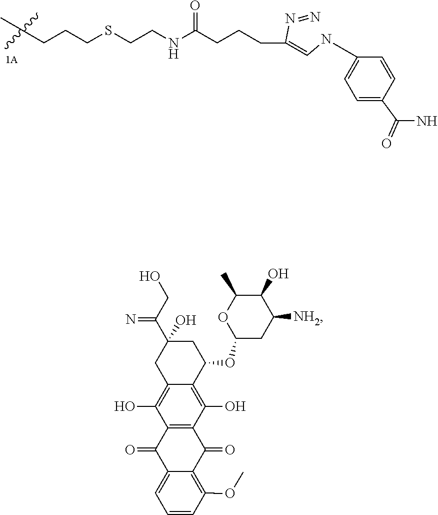

and wherein each bond 1A is connected to any —OH group in formula (II).

Example 29 relates to the pharmaceutical composition according to Example 27 or 28, wherein the compound of formula (II) has a molecular weight (Mw) of greater than about 5 kDa.

Example 30 relates to the pharmaceutical composition according to any one of Examples 27 to 29, wherein the anthracycline comprises doxorubicin.

Example 31 relates to the pharmaceutical composition of any one of claims 27 to 30, wherein the anthracycline is released from the composition at a pH of about 5.5 or below.

While multiple embodiments are disclosed, still other embodiments of the disclosure will become apparent to those skilled in the art from the following detailed description, which shows and describes illustrative embodiments of the disclosure. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF DRAWINGS

FIG. 1 shows a prior art attachment of doxorubicin to a mannosylated amine dextran (MAD) polymer. In this method, the hydrazone is attached to the mannosylated amine dextran polymer and that doxorubicin is added under highly acidic conditions to create the final conjugate structure shown on the right of the figure. The ball structure in this figure represents a mannosylated amine dextran.

FIG. 2 shows the derivatization of doxorubicin via condensation with 4-azidobenzohydrazide (hydrazide) forming doxorubicin hydrazone azide (DOX-hydrazone azide).

FIG. 3 Synthesis of a mannosylated alkyne dextran. The amino terminated leashes of MAD are modified through the addition of hexynoic acid to form a mannosylated alkyne dextran. The alkynes are the reacting partner for the doxorubicin hydrazone azide (FIG. 2) that form the conjugated product.

FIG. 4 shows synthesis of the ligand, which is a ligand for the copper ion catalyst and facilitates the CLICK addition of the doxorubicin hydrazone azide and the mannosylated alkyne dextran to produce the final product.

FIG. 5 shows an embodiment of a scheme showing the synthesis of a MAD construct carrying a doxorubicin payload (MAN-DOX). The chemical structure shown on the left of the figure is a derivative of doxorubicin modified to include a hydrazone azide attached to carbon 13. The derivatized doxorubicin is reacted with a mannosylated alkyne dextran in a copper-catalyzed reaction (CLICK) using a ligand to create the final conjugate product shown on the right of the figure. The mannosylated alkyne dextran is represented in FIG. 5 by the ball structure with the attached alkyne moiety.

FIG. 6 shows an embodiment of the synthesis pathways leading to the creation of the final product, MAN-DOX, shown on the right of the figure. Besides having a different synthesis pathway for creating the hydrazone linker, the final structure contains a hydrazone linker that has a significantly different structure (composition of matter) than that of the hydrazone linker disclosed in the prior art (FIG. 1).

FIG. 7 shows an embodiment of a scheme for the acid catalyzed hydrolysis of the hydrazone linker in the final product to release free doxorubicin. The figure describes how doxorubicin is released from the MAN-DOX construct in mildly acidic aqueous conditions of endosomes to which MAN-DOX is localized after binding to CD206.

FIG. 8 shows a table of the release of doxorubicin from a 10 kDa MAN-DOX construct in pH 7.4 Buffer Over Time.

FIG. 9 shows a table of the release of doxorubicin from a 10 kDa MAN-DOX construct in pH 5.5 Buffer Over Time.

FIG. 10 shows a table of the release of doxorubicin from a 3.5 kDa MAN-DOX construct in pH 4.65 and pH 7.4 Buffer Over Time.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed. Further, the term “about,” as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. Whether or not modified by the term “about,” the claims include equivalents to the quantities.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. In is also contemplated that, in certain aspects, unless expressly indicated to the contrary, individual substituents can be further optionally substituted (i.e., further substituted or unsubstituted).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed, that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific embodiment or combination of embodiments of the methods of the disclosure.

As used herein, the term “pharmaceutically acceptable carrier,” “pharmaceutically effective carrier,” or “carrier” refers to sterile aqueous or nonaqueous solutions, colloids, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of between about 1 to about 200 nm.

According to certain embodiments, the disclosures provided herein describe an improved chemical synthesis strategy, methods, and a resulting composition of matter related to conjugating anthracycline payloads through an acid labile hydrazone linker to carbohydrate polymers modified with amino terminated leashes (amine dextrans or amine carbohydrate polymers). The disclosed synthesis methods permit robust and reproducible conjugation of an anthracycline, such as, but not limited to doxorubicin, to the terminal amines on amine carbohydrate polymers and significantly reduces or eliminates undesirable intra- and intermolecular crosslinking that occurs in prior art methods of adding doxorubicin to carbohydrate polymers. It is understood that the chemical pathways described herein for the doxorubicin drug payload can be adapted to additional chemically related anthracyclines and a wide variety of carbohydrate polymeric backbones.

The compositions described herein possesses the features of targeted delivery and selective release of a drug payload. While the present disclosure is not limited to any particular mechanism of action, it is contemplated that, in some embodiments, the compositions of the disclosure bind tightly to a receptor displayed on its target cells whereupon it is internalized to endosomes. The endosomes in the target cells then become acidified. The acidic environment of the endosome s promotes the release of the payload inside the target cell.

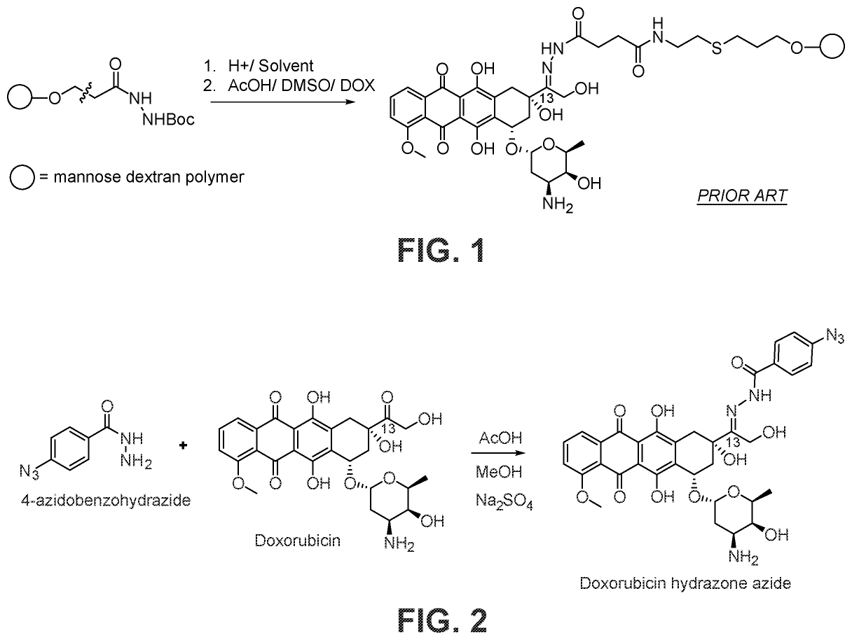

In addition, the disclosed synthesis method may remedy deficiencies in the prior art wherein the synthesis of a similar and related molecular construct results in molecular crosslinking and oligomerization, imparting undesired attributes to the final synthesis product. Crosslinking causes oligomerization (i.e., intermolecular crosslinking) that creates multimeric molecular products with significantly higher molecular weights. As would be appreciated by one having ordinary skill in the art, smaller molecular weight drug delivery vehicles exit the blood flow and penetrate target tissues more effectively than larger, heavier molecular constructs. Thus, preventing crosslinking increases the efficacy of drug delivery. An example of a prior art process is illustrated in FIG. 1. FIG. 1 shows a prior art method of doxorubicin-hydrazone formation on a dextran backbone.

The synthesis method and resulting compositions of the disclosure as described herein overcome the deficiencies of current methods of attaching doxorubicin to a free amine of a MAD or other polymeric backbone. In embodiments, an alternative synthetic pathway based on a copper-catalyzed, azide-alkyne click chemistry (CLICK) is utilized. In embodiments, the use of the CLICK chemistry results in a different hydrazone linker than linkers that may be contemplated by those skilled in the art. In aspects, an alkyne moiety is bound directly or indirectly to a carbohydrate polymer backbone, and an azide moiety is bound to doxorubicin via a degradable hydrazone. In aspects, any remaining azide may be removed from the product via diafiltration. It is recognized that in some embodiments, the reverse order would be amenable to CLICK chemistry where the azide moiety is directly or indirectly appended to the carbohydrate polymer backbone and the alkyne moiety is bound directly or indirectly to an anthracycline-hydrazone component. In further aspects, the reverse order would be amenable to CLICK chemistry where a hydrazone azide moiety is bound directly or indirectly to the carbohydrate polymer backbone, and the alkyne moiety is bound directly or indirectly to an anthracycline.

It should be noted that the hydrazone, hydrazide, azide, and alkyne are not limited to the specific example structures presented herein. These moieties may be bound to aromatic and/or alkyl groups of various ring sizes, chain lengths and physiochemical properties. In certain embodiments described herein, the hydrazone linkage synthesis with doxorubicin is conducted prior to coupling to dextran and does not involve exposure of the dextran backbone to acidic conditions, which may cause acid-based hydrolysis of the dextran backbone.

Further, in aspects, the synthesis methods described herein are more reproducible than prior art methods and allow for precise and predictable loading of doxorubicin based on stoichiometry of the coupling components. As a result, the chemical method and synthesis strategies described herein are superior to compositions and methods described in the prior art. In aspects, the compounds, compositions, and methods provided herein do not facilitate the molecular crosslinking observed in the chemistry of the prior art, thus, providing for a drug delivery construct with a more narrowly defined and smaller molecular weight (Mw).

Further, prior known methods and resulting products (shown for example in FIG. 1) are not the most desirable for use in pharmaceutical products, as they lead to molecular crosslinking, have low reproducibility, and are difficult to reproduce the loading of drug payloads, such as, but not limited to, doxorubicin. These deficiencies in the prior art are remedied by the methods and compositions described in this disclosure. In some aspects, provided herein are methods for conjugating anthracycline to a polymeric backbone or carrier. In further aspects, provided herein are methods for synthesizing an anthracycline-carbohydrate polymer construct.

In certain embodiments, disclosed herein is a method for conjugating anthracycline to a carbohydrate polymer comprising, providing (1) a carbohydrate polymer comprising at least one alkyne moiety, and (2) an anthracycline derivatized to a hydrazone azide. In embodiments, the alkyne moiety is bound directly or indirectly to the carbohydrate polymer. In alternative embodiments, the method for conjugating anthracycline to a carbohydrate polymer comprises providing (1) a carbohydrate polymer comprising at least one hydrazone azide moiety, and (2) an anthracycline comprising at least one alkyne moiety. In embodiments, at least one hydrazone azide moiety is bound directly or indirectly to the carbohydrate polymer. In further embodiments, the alkyne moiety may be bound directly or indirectly to the anthracycline. In some aspects, the method further comprises a step of reacting the carbohydrate polymer with the anthracycline in the presence of a Cu¹ catalyst and an amine ligand to form a 1,3-triazole linkage between the anthracycline and the carbohydrate polymer. In some aspects, the linkage results from a 1,3-cycloaddition between the azide and the alkyne.

In certain embodiments, the disclosed method provides for the conjugation of doxorubicin to an aminated carbohydrate polymer backbone. In further embodiments, the disclosed method provides for a method of ligating doxorubicin to a carbohydrate polymer. In certain implementations of these embodiments, the disclosed methods include the steps of providing an alkyne carbohydrate polymer; providing a doxorubicin hydrazone azide; reacting the alkyne dextran with the doxorubicin hydrazone azide in the presence of a Cu¹ catalyst and a ligand to form a 1,3-triazole linkage between the doxorubicin and carbohydrate polymer; and wherein the linkage results from a 1,3-cycloaddition between the azide of the doxorubicin hydrazone azide and the alkyne of the alkyne carbohydrate polymer.

In further embodiments, methods of synthesizing an anthracycline-carbohydrate polymer construct are provided. In aspects, the method comprises providing an alkyne carbohydrate polymer. In certain embodiments, the step of providing the alkyne carbohydrate polymer comprises methods for preparing the alkyne carbohydrate polymer from an amine carbohydrate polymer. In certain aspects, the preparation of the alkyne carbohydrate polymer starts with employing a carrier construct comprising a carbohydrate polymer backbone having conjugated thereto mannose-binding C-type lectin receptor targeting moieties. In further embodiments, the carrier construct further comprises one or more leashes for attaching the mannose-binding C-type lectin receptor targeting moieties to the carbohydrate polymer backbone. In some aspects, one or more additional moieties may be present between the leash and the mannose-binding C-type lectin receptor targeting moieties. In further embodiments, the leash is not attached to a mannose-binding C-type lectin receptor targeting moiety, and instead, is provided as a standalone leash attached to the carbohydrate polymer backbone. In embodiments, the leash may be an amino terminated leash as further described herein.

In some embodiments, the mannose-binding C-type lectin receptor targeting moiety comprises a mannosyl coupling reagent, mannose, high-mannose glycans or mannose oligosaccharides, fucose, n-acetylglucosamine, peptides, galactose or a combination thereof. In some embodiments, the mannose-binding C-type lectin receptor targeting moiety comprises mannose, fucose, and n-acetylglucosamine. In further embodiments, the mannose-binding C-type lectin receptor targeting moiety is mannose. Examples of such constructs include mannosylated amine dextrans (MADs), which comprise a dextran backbone having mannose molecules conjugated to glucose residues of the backbone and having an active pharmaceutical ingredient conjugated to glucose residues of the backbone. Tilmanocept is a specific example of a MAD. A tilmanocept derivative that is tilmanocept without DTPA conjugated thereto is a further example of a MAD.

MADs are synthetic molecules purposefully designed to be high affinity ligands for mannose-binding C-lectin type receptors, such as, for example, CD206. MADs have been described in U.S. Pat. No. 6,409,990 (990 patent), which is hereby incorporated by reference in its entirety. Thus, the backbone comprises a plurality of glucose moieties (i.e., residues or subunits) primarily linked by α-1,6 glycosidic bonds. Other linkages such as α-1,4 and/or α-1,3 bonds may also be present.

In embodiments, the backbone comprises a carbohydrate polymer. Examples of suitable carbohydrate polymers include, but are not limited to, cellulose, dextran, and mannan. Further embodiments may comprise a backbone that is not a dextran backbone. Some embodiments may have a monosaccharide-based backbone that does not comprise dextran. The backbone of carbohydrate-based carrier molecules described herein can comprise a glycan other than dextran, wherein the glycan comprises a plurality of monosaccharide residues (i.e., sugar residues or modified sugar residues). In certain embodiments, the glycan backbone has sufficient monosaccharide residues, as well as optional groups such as one or more amino acids, polypeptides and/or lipids, to provide a MW of about 1 to about 50 kDa. As would be appreciated by the skilled artisan when considering the disclosure contained herein, when referring to a “dextran” backbone, other monosaccharide residues may be considered to be substituted in compounds described herein. Additional descriptions of carbohydrate-backbone-based carrier molecules used for targeting CD206 are described in PCT application No. PCT/US2017/055211, which is incorporated herein by reference in its entirety.

The size of a MAD or amine carbohydrate polymer construct can be varied by changing the size of the initial carbohydrate polymer upon which the MAD construct or amine carbohydrate polymer construct is assembled. In some embodiments, the carbohydrate polymer backbone is between about 1 kDa and 100 kDa. The dextran-based moiety may be at least about 50 kDa, at least about 60 kDa, at least about 70 kDa, at least about 80 kDa, or at least about 90 kDa. The dextran-based moiety may be less than about 100 kDa, less than about 90 kDa, less than about 80 kDa, less than about 70 kDa, or less than about 60 kDa. Alternatively, in some embodiments, the dextran backbone has a MW of between about 1 kDa and about 50 kDa, while in other embodiments the dextran backbone has a MW of between about 3 kDa and about 25 kDa. In still other embodiments, the dextran backbone has a MW of between about 8 kDa and about 15 kDa, such as about 10 kDa. While in other embodiments the dextran backbone has a MW of between about 1 kDa and about 5 kDa, such as about 3 kDa. Beneficially, the smaller sizes of the disclosed constructs enable greater tumor penetration and greater localization to tumor associated macrophages (TAMs) than is possible with other larger constructs.

In certain embodiments, the mannose-binding C-type lectin targeting moieties are attached to between about 15% and about 100%, between about 17% and about 65%, or about 20% and about 60% of the glucose residues via the amino terminated leashes. In further embodiments, the mannose-binding C-type lectin targeting moieties are attached to up to about 60%, up to about 70%, up to about 80%, up to about 90%, or up to about 100% of the glucose residues via the amino terminated leashes. In certain aspects, the percentages may vary depending on the size of the carbohydrate polymer backbone.

In aspects, the leash may be attached to from about 30% to about 100% of the backbone moieties, or from about 70% to about 90% of the backbone moieties. The leashes may be the same or different. In some embodiments, the leash is an amino terminated leash. In some embodiments, the leashes may comprise the formula —(CH₂)ₚS(CH₂)_{q}—NH—, wherein p and q are integers from 0 to 5. In further embodiments, the leash comprises the formula —(CH₂)₃S(CH₂)₂NH—. In embodiments where the leash is not attached to a mannose-binding C-type lectin receptor targeting moiety, the leash may comprise the formula —(CH₂)ₚS(CH₂)_{q}—NH₂, wherein p and q are integers from 0 to 5.

In some embodiments, the leash may be a chain of from about 1 to about 20 member atoms selected from carbon, oxygen, sulfur, nitrogen and phosphorus. The leash may be straight chain or branched. The leash may also be substituted with one or more substituents including, but not limited to, halo groups, perfluoroalkyl groups, perfluoroalkoxy groups, alkyl groups, such C₁₋₄ alkyl, alkenyl groups, such as C₁₋₄ alkenyl, alkynyl groups, such as C₁₋₄ alkynyl, hydroxy groups, oxo groups, mercapto groups, alkylthio groups, alkoxy groups, nitro groups, azidealkyl groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkylcarbonyloxy groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, —NH—NH₂; ═N—H; ═N— alkyl; —SH; —S-alkyl; —NH—C(O)—; —NH—C(═N)— and the like. As would be apparent to one skilled in the art, other suitable leashes are possible. In some embodiments, the mannose-binding C-type lectin targeting moieties may be conjugated to the amino groups of the amino terminated leash via an amidine and/or amide linker.

In certain implementations of these embodiments, the method involves converting a portion of the primary amines in the amine carbohydrate polymer to amides with at least one alkyne moiety under dehydrative conditions to form the alkyne carbohydrate polymer. In certain implementations, the alkyne moiety comprises an alkynoic acid. In some embodiments, the alkyne is hexynoic acid. As will be appreciated, various methods of achieving activation are possible. Examples include, but are not limited to use of reactive esters, halides, and or anhydrides. In aspects, a mannosylated-alkyne dextran is synthesized between the amine carbohydrate polymer and alkyne moiety. In certain embodiments, the reaction is carried out at a pH of at least about 6.5. In further embodiments, the reaction is carried out a pH of between about 6.5 and about 10, between about 7 and about 9, and between about 8 and 9. In further embodiments, the reaction is carried out at a pH of between about 8.3 to about 8.5.

In certain embodiments, the mannosylated-alkyne dextran has the following structure of formula (I):

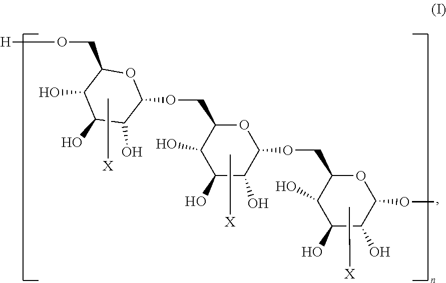

wherein
each X is independently H, L2-R, or L3-Y;
each L2 and L3 are independently leashes;
each R independently comprises a mannose-binding C-type lectin receptor targeting moiety, or H;
each Y independently comprises a terminal alkyne moiety, or H; and
n is an integer greater than zero,
wherein each unit of n may be the same or different.

In some aspects, the leashes may be any leash as further described herein. In some embodiments, the leashes are amino terminated leashes, wherein the amino terminated leashes may comprise the formula —(CH₂)ₚS(CH₂)_{q}—NH—, wherein p and q are integers from 0 to 5.

In some aspects, at least one R comprises a mannose-binding C-type lectin receptor targeting moiety. Suitable mannose-binding C-type lectin receptor targeting moieties are as described herein. In some embodiments, the mannose-binding C-type lectin receptor targeting moiety selected from the group consisting of mannose, fucose, and n-acetylglucosamine.

In some aspects, at least one Y comprises the terminal alkyne moiety. In aspects, the terminal alkyne moiety is the alkyne moiety attached to the alkyne carbohydrate polymer as further described herein.

In some aspects, n is an integer greater than zero. In other aspects, n is an integer greater than 1. In further aspects, n may be an integer between 1 and about 50, between about 5 and about 40, or between about 5 and about 30. As would be understood by those skilled in the art, each unit of n may be the same or may be different. As each X may independently be H, L2-R, or L3-Y, each unit of n may consist of any combination of X selected from H, L2-R, or L3-Y.

In some embodiments, X may be

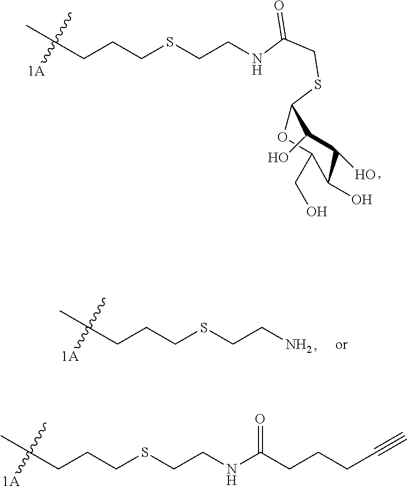

wherein each bond 1A is connected to any —OH group in formula (I). For example, an example unit of n may have the following structure:

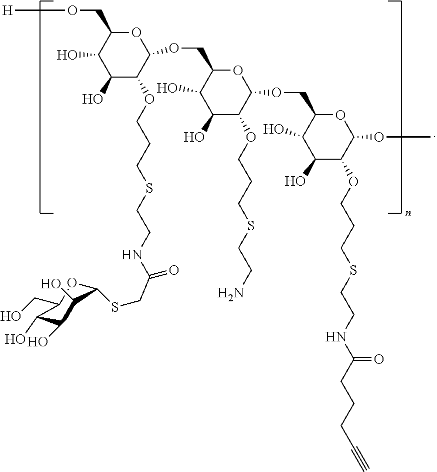

However, as would be appreciated by those skilled in the art, additional units of n may not consist of

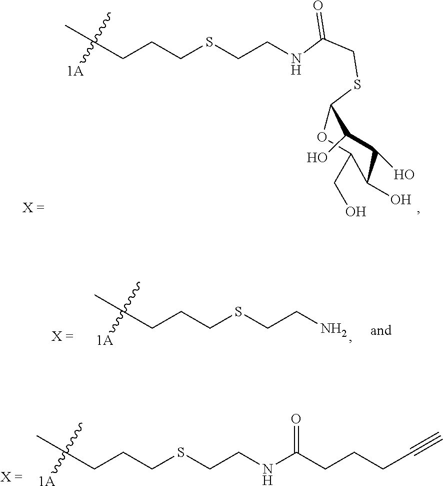

in the exact same order. Each X may be selected from any one of H, L2-R, or L3-Y. As further described herein, it will be appreciated that the amide linker between the amino-terminated leash and the mannose-binding C-type lectin receptor targeting moiety may comprise an amide linker (as shown), an amidine linker, or a combination thereof.

In aspects, the methods comprise providing an anthracycline hydrazone azide. In certain embodiments, the step of providing the anthracycline hydrazone azide further comprises the step of derivatizing the anthracycline by reacting the anthracycline with a hydrazide via condensation to form the anthracycline hydrazone azide. In some aspects, any hydrazide moiety may be used such that the hydrazide comprises an azide moiety and a hydrazone moiety. In some embodiments, the hydrazide comprises an aryl group. In other embodiments, the hydrazide comprises a substituted or unsubstituted, branched or unbranched alkyl group. In certain embodiments, the hydrazide is 4-azidobenzohydrazide. In embodiments, the anthracycline may be one or more of daunorubicin, doxorubicin, epirubicin, idarubicin, mitoxantrone, pirarubicin, aclarubicin, or valrubicin. In further embodiments, the anthracycline may be doxorubicin. In an example embodiment, doxorubicin may be modified with the hydrazide by attaching the hydrazide to carbon 13 as shown in FIG. 2.

In aspects, the methods further comprise reacting an alkyne carbohydrate polymer with an anthracycline hydrazone azide in the presence of a Cu¹ catalyst and a ligand. In some aspects, the reaction of the anthracycline hydrazone azide and alkyne carbohydrate polymer in the presence of a Cu¹ catalyst and ligand forms a 1,3-triazole linkage between the anthracycline and carbohydrate polymer. In aspects, the 1,3-triazole linkage results from a 1,3-cycloaddition between the azide of the anthracycline hydrazone azide and the alkyne of the alkyne carbohydrate polymer. The reaction process between the azide and alkyne moieties may be further referred to herein as click chemistry (CLICK).

In some embodiments, the ligand comprises a compound capable of binding Cu. Use of the ligand serves to prevent the degradation of available Cu¹ via redox and disproportionation pathways. In certain embodiments, the Cu operates as a catalyst. In aspects, the catalyst comprises a Cu¹ catalyst. In certain embodiments, the ligand is an amine ligand (e.g. the ligand has one or more amine groups). In certain embodiments, the amine ligand has the following structure:

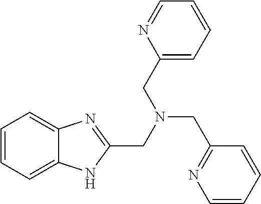 [SMILES: c1ccc(CN(Cc2ccccn2)Cc2nc3ccccc3[nH]2)nc1]

In certain embodiments, the method further provides steps for synthesizing the amine ligand. In certain implementations, the ligand is synthesized through the reaction of ((2-benzimidazolyl)methyl)amine dihydrochloride and 2-(chloromethyl)pyridine in the presence of Et₃N. In further embodiments, the ligan d may comprise tris(heterocycle-methyl)amines containing a combination of two or more benzimidazole or pyridyl groups. One skilled in the art will appreciate that other ligands and synthesis methods are possible.

In exemplary implementations, the ligand and Cu¹ catalyst are present at a ratio of between about 1:5 to about 5:1, between about 1:2 to about 2:1, or about 1:1. According to certain alternative embodiments, the foregoing reaction is carried out without the use of a ligand.

According to certain embodiments, the step of reacting the alkyne carbohydrate polymer with the anthracycline hydrazone azide is carried out in the presence of sodium ascorbate. In exemplary implementations of these embodiments, the Cu¹ catalyst and ascorbate are present at a ratio of between about 1:5 to about 9:10, between about 1:3 to about 7:10, between about 3:4 to about 3:6, or about 3:5.

In certain embodiments, the alkyne moiety and Cu¹ catalyst are present at a ratio of between about 1:1 to about 5:1, between about 1:1 to about 3:1, or about 2:1.

According to further embodiments, the reaction is performed in the presence of DMSO and water. In exemplary implementations, DMSO and water are present at a ratio of between about 4:1 to about 20:1, between about 6:1 to about 12:1, between about 6:1 to about 8:1, or about 7.5:1.

In embodiments, the reaction between the alkyne carbohydrate polymer with the anthracycline hydrazone azide in the presence of a Cu¹ catalyst and a ligand forms an anthracycline-carbohydrate polymer construct. In certain embodiments, the anthracycline-carbohydrate polymer has the structure of formula (II):

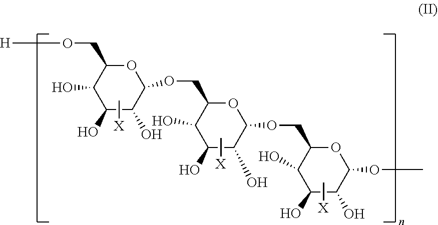

wherein
each X is independently H, L1-A, L2-R, or L3-Y;
each L1, L2 and L3 are independently leashes;
each A independently comprises an anthracycline hydrazone azide moiety, or H;
each R independently comprises a mannose-binding C-type lectin receptor targeting moiety, or H;
each Y independently comprises a terminal alkyne moiety, or H; and
n is an integer greater than zero,
wherein each unit of n may be the same or different.

In some aspects, the leashes may be any leash as further described herein. In some embodiments, the leashes are amino terminated leashes, wherein the amino terminated leashes may comprise the formula —(CH₂)ₚS(CH₂)_{q}—NH—, wherein p and q are integers from 0 to 5.

In some aspects, at least one A comprises the anthracycline hydrazone azide moiety. Suitable anthracycline hydrazone azide moieties are described herein. In some embodiments, the anthracycline comprises doxorubicin.

In some aspects, at least one R comprises a mannose-binding C-type lectin receptor targeting moiety. Suitable mannose-binding C-type lectin receptor targeting moieties are as described herein. In some embodiments, the mannose-binding C-type lectin receptor targeting moiety selected from the group consisting of mannose, fucose, and n-acetylglucosamine.

In some aspects, at least one Y comprises the terminal alkyne moiety. In aspects, the terminal alkyne moiety is the alkyne moiety attached to the alkyne carbohydrate polymer as further described herein. In further aspects, the anthracycline-carbohydrate polymer does not contain leashes with terminal alkyne moieties due to the CLICK reaction with the anthracycline hydrazone azide moiety.

In some aspects, n is an integer greater than zero. In other aspects, n is an integer greater than 1. In further aspects, n may be an integer between 1 and about 50, between about 5 and about 40, or between about 5 and about 30. As would be understood by those skilled in the art, each unit of n may be the same or may be different. As each X may independently be H, L1-A, L2-R, or L3-Y, each unit of n may consist of any combination of X selected from H, L1-A, L2-R, or L3-Y.

In some embodiments, X may be H,

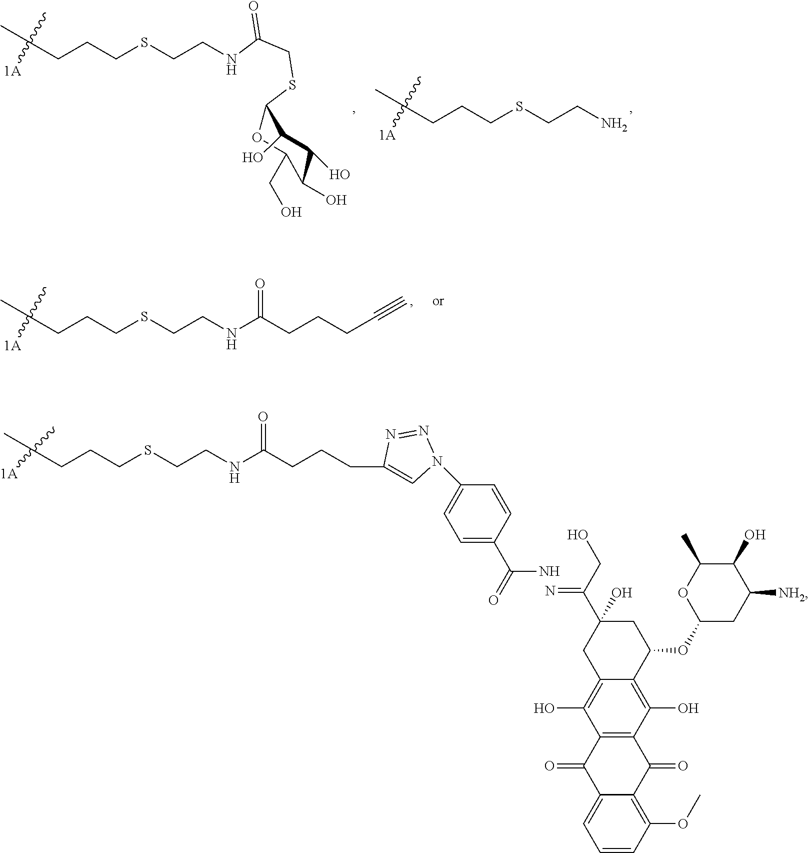

wherein each bond 1A is connected to any —OH group in formula (II). For example, an example unit of n may have the following structure:

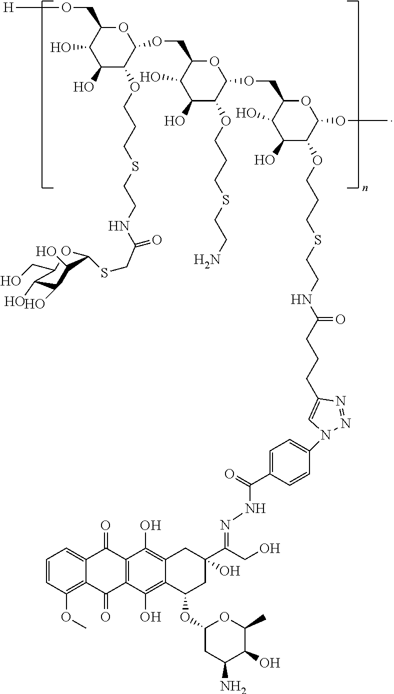

However, as would be appreciated by those skilled in the art, additional units of n may not consist of X═

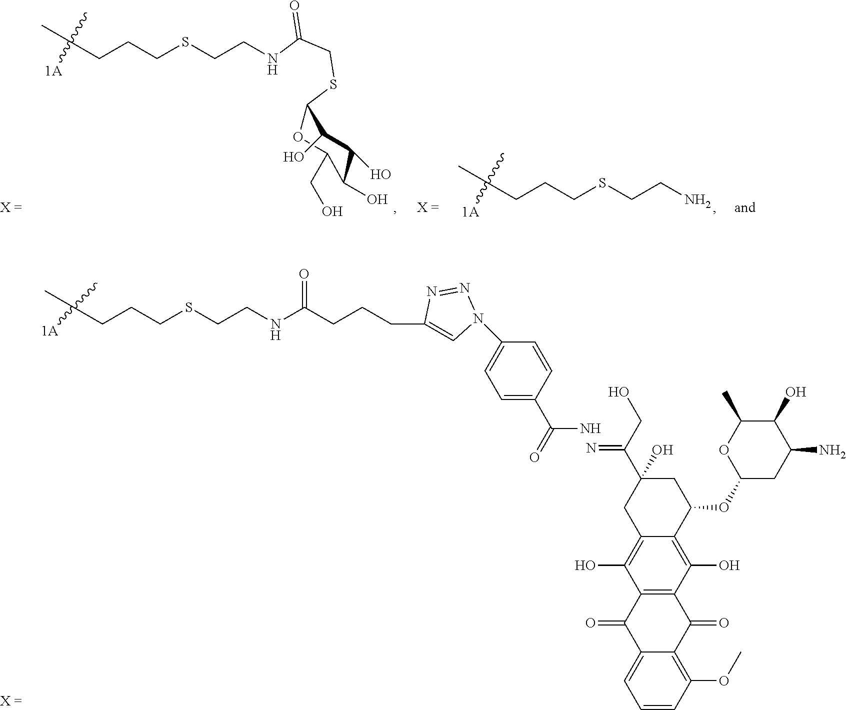

in the exact same order. Each X may be selected from any one of H, L1-A, L2-R, or L3-Y. As further described herein, it will be appreciated that the amide linker between the amino-terminated leash and the mannose-binding C-type lectin receptor targeting moiety may comprise an amide linker (as shown), an amidine linker, or a combination thereof.

Further disclosed herein are pharmaceutical compositions comprising a compound having the structure of formula (II) as described herein and a pharmaceutically acceptable carrier thereof.

According to certain embodiments, the anthracycline of the anthracycline-carbohydrate polymer construct may be released from the pharmaceutical composition at a suitable pH. In some embodiments, the pH is at a pH level of about 6 or below, about 5.5 or below, or about 5 or below. Beneficially, the anthracycline-carbohydrate polymer constructs are able to release the anthracycline payload at acidic conditions for targeted delivery of the therapeutic.

In certain embodiments the composition is a mannosylated amine carbohydrate polymer. In further embodiments, the composition is a mannosylated amine dextran (MAD backbone) configured as a macrophage mannose receptor (CD206) targeted drug delivery vehicle intended to ablate and/or phenotypically alter CD206 expressing cells. In further embodiments, the composition is a MAD purposefully designed to be high affinity ligands for mannose-binding C-type lectin receptors. Using the MAD backbone, a wide variety of small molecule payloads or diagnostic imaging moieties can be specifically delivered to CD206 expressing cells.

CD206 is expressed by macrophages, macrophage-like cells such as Kupffer cells of the liver and microglia of the brain, and subsets of dendritic cells (DC) and myeloid derived suppressor cells (MDSC). All of these cell types are important components of innate immunity. CD206 is also expressed by mesangial cells of the kidneys. Mesangial cells have features of both macrophages and smooth muscle cells. At sites of inflammation, macrophages can adopt a wide variety of activated phenotypes depending on signals received by individual macrophages in their local immune microenvironment. Depending upon which signals from its microenvironment a macrophage is responding to, an individual macrophage will become activated by altering (increasing or decreasing) their expression of many hundreds of genes. One gene that is frequently increased in expression upon macrophage activation is CD206. Macrophage phenotypes are known to be alterable and adaptable to changes in signals to which the macrophage is responding.

Activated macrophages are numerous in, and important contributors to, the inflammatory microenvironments of many societally important illnesses with chronic inflammation as a component of disease. Examples of societally important illnesses with macrophage involved chronic inflammation include, but are not limited to cancer (including, but not limited to, Kaposi's Sarcoma), atherosclerosis (e.g. cardiovascular disease and stroke), and rheumatoid arthritis (RA). In these conditions and others, activated macrophages act both as direct effectors of pathology and, in conjunction with DC and MDSC that usually express CD206, as regulators of the phenotypic states of cells of the adaptive immune response (i.e. lymphocytes). Activated macrophages can be pro-inflammatory, directly attacking targets and stimulating immune responses by lymphocytes, or alternatively, can promote wound healing and immunosuppression, suppressing the adaptive immune response. Ablating or phenotypically altering macrophages is recognized as a therapeutic strategy to change/improve the course of illnesses with macrophage, and possibly DC and MDSC, involved chronic inflammation as contributing causes.

CD206+ macrophages are host cells for various pathogens. Examples of human pathogens for which CD206+ macrophages are host cells are dengue virus, yellow fever virus, leishmanial parasites, and Mycobacterium tuberculosis. In addition, human immunodeficiency virus (HIV) infects CD206+ macrophages and may use these CD206+ hosts cells as a drug resistant reservoir to avoid elimination mediated by combined anti-retroviral therapy.

Further disclosed herein is a method for addition of doxorubicin to MAD-based drug delivery vehicles that deliver their payloads specifically to CD206 expressing cells. The disclosed compositions have various attributes that contribute to their efficacy as drug delivery vehicles. These attributes include, among others: the ability to retain their doxorubicin payloads at physiological pH (i.e. pH=7.4) but release their payloads in the mildly acidic conditions encountered in endosomes; minimized molecular crosslinking during addition of doxorubicin, leading to synthesis products with smaller average molecular weights (Mw); and the ability to load MAN-based constructs with greater numbers of doxorubicin moieties and to reproducibly load the constructs with the same number of doxorubicin moieties.

In some aspects, the compounds and compositions of the disclosure, or pharmaceutically acceptable salts thereof, may be combined as the active ingredient in an admixture with a pharmaceutical carrier according to conventional pharmaceutical compounding techniques. The carrier may take a wide variety of forms depending on the form of preparation desired for administration, e.g., parenteral (including intravenous), rectal, etc. Thus, the pharmaceutical compositions of the present disclosure may be presented as discrete units suitable for administration such as capsules, cachets or tablets each containing a predetermined amount of the active ingredient. Further, the compositions may be presented as a powder, as lyophilized powder, as granules, as a solution, as a suspension in an aqueous liquid, as a non-aqueous liquid, as an oil-in-water emulsion or as a water-in-oil liquid emulsion. In addition to the common dosage forms set out above, the compounds of the disclosure, and/or pharmaceutically acceptable salt(s) thereof, may also be administered by controlled release delivery devices. The compositions may be prepared by any of the methods of pharmacy. In general, such methods include a step of bringing into association the active ingredient with the carrier that constitutes one or more necessary ingredients. In some aspects, the compositions are prepared by uniformly and intimately admixing the active ingredient with the carrier(s). The product may then be conveniently shaped into the desired presentation.

In embodiments, the pharmaceutical compositions of the disclosure may include a pharmaceutically acceptable carrier and either a compound of the disclosure described herein or a pharmaceutically acceptable salt of the compounds of the disclosure. The compounds, or pharmaceutically acceptable salts thereof, may also be included in pharmaceutical compositions in combination with one or more other therapeutically active compounds.

In aspects, the pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

The pharmaceutical compositions of the present disclosure comprise a compound of the disclosure (or pharmaceutically acceptable salts thereof) as an active ingredient, a pharmaceutically acceptable carrier, and optionally one or more additional therapeutic agents or adjuvants. The instant compositions include compositions suitable for rectal and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present disclosure suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringeability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of this disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the disclosure, and/or pharmaceutically acceptable salts thereof, can also be prepared in powder or liquid concentrate form.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

### EXAMPLES

The following examples are put forth so as to provide those of ordinary skill in the art with a complete disclosure and description of certain examples of how the compounds, compositions, articles, devices and/or methods claimed herein are made and evaluated and are intended to be purely exemplary of the disclosure and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the disclosure.

### Example 1

### Preparing the Doxorubicin (DOX-Hydrazone) Azide (FIG. 2)

Procedure: In a round bottom flask, doxorubicin and the hydrazide were mixed in DMSO/MeOH and sodium sulfate was added. To the resulting mixture, 2 drops of acetic acid were added. The reaction was stirred for 48 hours over which time a red precipitate formed. After 48 hours, most of the starting hydrazide was consumed as determined by liquid chromatography mass spectrometry (LC/MS). The reaction was filtered by vacuum filtration. The vacuum was turned off and a minimum amount of DMSO was added to the filter cake to dissolve the red precipitate. The vacuum was applied to pull the DMSO solution through the filter and the filter was washed with methanol to chase the remaining product/DMSO into the collection flask. The volatiles were removed by vacuum evaporation and methylene chloride was added. The mixture was cooled in a −20° C.° freezer overnight to precipitate the product. The product was removed by centrifugation or filtration, washed with methylene chloride, and dried in vacuo. FIG. 2 shows the scheme of an embodiment of the derivatization of doxorubicin via condensation with 4-azidobenzohydrazide as the hydrazide to form the DOX-hydrazone azide. This example yielded 273 mg (89% of theoretical yield) using the amounts set forth in Table 1.

TABLE 1

| | | Other | | | | |
|---|---|---|---|---|---|---|
| Compound | MW | Features | Amount | | Moles | Ratio |
| Hydrazide | 171.10 | | 75 | mg | 0.000438 | 1 |
| Doxorubicin | 579.98 | | 255 | mg | 0.000439 | 1 |
| Na₂SO₄ | 142.04 | | 750 | mg | 0.00528 | 12 |
| AcOH | | glacial | 2 | drops | | |
| MeOH | | | | | | 6 ml |
| DMSO | | | | | | 1 ml |

### Example 2

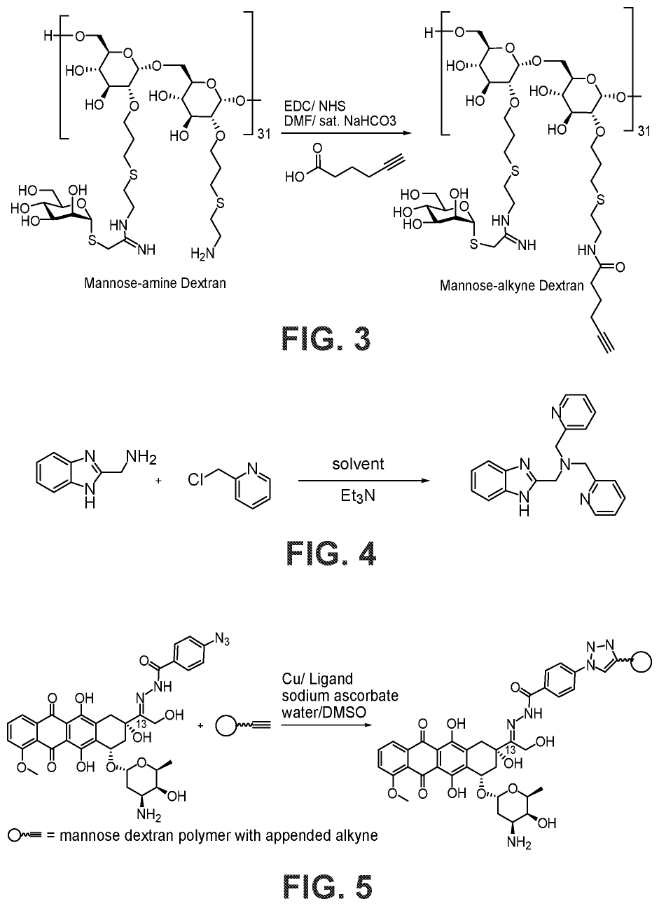

### Addition of Alkyne to Mannose-Amine Dextran on 10 kDa and 3.5 kDa Dextran (FIG. 3)

Mannose-alkyne dextran was prepared from mannose-amine dextran and hexynoic acid under carbodiimide coupling conditions. The synthesis of the MAD backbone itself is described in the '990 patent which has been hereby incorporated by reference in its entirety.

10 kDa Dextran Procedural Example: In a flask, 483 mg (4.31 mmol) of 1-hexynoic acid under nitrogen gas was dissolved in 6.2 ml of anhydrous dimethylformamide (DMF). 594 mg (5.16 mmol) of N-hydroxysuccinimide was added followed by 906 μL (5.12 mmol) of 1-Ethyl-3-(3-dimethylaminopropyl)carbodiimide. After stirring at room temperature for 45 minutes, 1.5 ml (1.05 mmol) of this activated ester solution was charged into an aqueous saturated sodium bicarbonate solution containing 2 g of mannosylated dextran (¹H NMR: Ave. 18-mannose, 20 amines, Mw 18,570 g/mol) at 25 mg/ml. The reaction was monitored by following the decrease in amine content in the reaction solution. In this example, an additional 2.0 ml (1.4 mmol) of the activated ester was charged to the amine dextran solution after 1 hour and transferred to a stirred-ultrafiltration cell fitted with a 3 kDa molecular weight cut-off (MWCO) membrane at 3 hours of total reaction time. The dextran reaction solution was concentrated to minimal volume and washed 3× with 150 ml of purified water via ultrafiltration concentration. The retentate was subsequently passed through a 0.45 μm filter, frozen and lyophilized providing mannosylated alkyne dextran as an off-white foam solid (1.75 g, ¹H NMR: Ave. 18 mannoses, 12 alkyne chains, 8 amines, 19,699 g/mol). FIG. 3 shows the scheme of an embodiment of the synthesis of the mannose-alkyne dextrans on a 10 kD dextran backbone.

3.5 kDa Dextran Procedural Example: In a flask, 670 mg (6.0 mmol) of 1-hexynoic acid under nitrogen gas was dissolved in 9 ml of anhydrous DMF. 830 mg (7.2 mmol) of N-hydroxysuccinimide was added followed by 1.38 g (7.2 mmol) of 1-Ethyl-3-(3-dimethylaminopropyl)carbodiimide hydrochloride. After stirring at room temperature for 45 minutes, 2.3 ml (1.53 mmol) of this activated ester solution was charged into an aqueous saturated sodium bicarbonate solution containing 1.18 g of mannosylated dextran (¹H NMR: Ave. 10-mannose, 10 amines, Mw 8,196 g/mol) at 25 mg/ml. The reaction was monitored by following the decrease in amine content in the reaction solution. After the desired loading of alkyne was achieved, the reaction solution was transferred to a stirred-cell fitted with a 3 kDa MWCO membrane. The dextran reaction solution was concentrated to minimal volume and washed three times with 150 ml of purified water via ultrafiltration concentration. The retentate was passed through a 0.45 μm filter, frozen and lyophilized providing mannosylated alkyne dextran as an off-white foam solid (1.11 g, 1H NMR: Ave. 10 mannoses, 10 alkyne chains, 9,137 g/mol).

### Example 3

Synthesis of the ((2-benzimidazolyl)methyl)-bis-((2-pyridyl)methyl)amine (ligand) (FIG. 4)

FIG. 4 shows an embodiment of the synthesis of a copper ligand for use in a copper-catalyzed azide-alkyne CLICK chemistry reaction. Click coupling results in the formation of a 1,3-triazole via 1,3-cycloaddition. The triazole is formed from an azide and an alkyne and is catalyzed by copper in the +1 oxidation state. These transformations are accelerated by amine ligands, primarily by preventing the degradation of available Cu¹ via redox and disproportionation pathways. Click couplings using the commercially available tris[(1-benzyl-1H-1,2,3-triazol-4-yl)methyl]amine ligand resulted in low yields, so ((2-benzimidazolyl)methyl)-bis-((2-pyridyl)methyl)amine was prepared as a ligand for copper which worked satisfactorily in the CLICK coupling. Further guidance is provided by Presolski, S. I. et al. J. Am. Chem. Soc. 2010, 132, 14570-14576., which is hereby incorporated by reference in its entirety.

Procedure: In a round bottom flask 0.440 g (2 mmol) of ((2-benzimidazolyl)methyl)amine dihydrochloride and 0.689 g (4.2 mmol) of 2-(chloromethyl)pyridine were dissolved in 80 ml of CH₂Cl₂ and cooled to 0° C. 1.8 ml (13 mmol) of Et₃N dissolved in 10 ml of CH₂Cl₂ was added dropwise at 0° C. and allowed to warm to room temperature. The reaction was allowed to stir at room temperature for 4 days after which the reaction was diluted by addition of 60 ml of CH₂C₁₂. The combined organics were washed with 50 ml of water and condensed in vacuo. The resulting material was purified by flash chromatography (Biotage, MeOH/EtOAc gradient on neutral alumina) followed by recrystallization from CCl₄. The process yielded 0.22 g (34% of theoretical yield) of a light brown hygroscopic solid.

### Example 4

### CLICK Coupling of Doxorubicin Hydrazone Azide to Mannose-alkyne Dextran (FIG. 5) 10 kDa Dextran

Procedure: Copper (II) sulfate, sodium ascorbate and water were combined in a screw cap vial and allowed to stir for 2-5 minutes at room temperature. In a separate vial the mannose-alkyne dextran, DOX-hydrazone azide, and copperligand from Examples 1-3 were combined in DMSO and stirred for 3-4 minutes. Using a pipette, the contents of the copper/ascorbate vial were transferred to the stirring azide/alkyne vial and capped. The reaction continued stirring at room temperature for an additional 1.5-2 hours, then was diluted with 4 ml of deionized (DI) water and added to the top of a 10 kDa MWCO spin-filter. This was spun at 14,000 rpm until separated. A fresh aliquot of water was added, and the process repeated. The washing was completed for a total of three washes.

The retentate was removed using DI water. The retentate formed a sticky solid which slowly dissolved. A mechanical aid (needle or spatula) was used to dislodge the solids and remove the retentate. In a falcon tube, the solid retentate was dissolved in DI water using mild heat, sonication, and vortexing. The solution was then centrifuged and decanted, leaving a solid residue in the tube. This process was repeated two more times to recover a maximum amount of soluble product. Most of the product was in solution and only a small pellet remained. The pellet was discarded and the product solution lyophilized. The resultant product was weighed and analyzed for free and bound doxorubicin. FIG. 5 shows a scheme of an embodiment for appending the DOX-hydrazone azide to a mannose-alkyne dextran backbone utilizing a copper-catalyzed reaction through a ligand. Table 2 sets forth the compounds and their amounts and characteristics for this example.

TABLE 2

| | MW | Other | | | | |
|---|---|---|---|---|---|---|
| Compound | (g/mol) | Features | Amount | | Moles | Ratio |
| DOX- | 702 | | 13 | mg | 0.00001851 | 12 |
| hydrazone | | | | | | |
| Azide | | | | | | |
| Alkyne | 19,699 | 12 alkynes | 30 | mg | 0.00000153 | 1 |
| Copper sulfate | 159.6 | | 1.5 | mg | 0.000009398 | 6 |
| Ligand | 329.4 | | 3 | mg | 0.000009107 | 6 |
| Sodium | 198.11 | | 3 | mg | 0.00001514 | 10 |
| ascorbate | | | | | | |
| DMSO | | | 0.75 | ml | | 7.5 |
| Water | | | 0.1 | ml | | 1 |

This example had a yield of 333 mg (77.4%). Free doxorubicin was present at 0.91% by mass and bound doxorubicin was found to be at 6.7 DOX/chain (17.8% by mass).

### 3.5 kDa Dextran

Procedure: In a screw cap vial copper sulfate, sodium ascorbate and water were combined and stirred for 2-5 minutes. In a separate vial, the mannose-alkyne dextran, DOX-hydrazone azide, and copper ligand from Examples 1-3 were combined in DMSO. The contents of the azide/alkyne vial were allowed to stir for 3-4 minutes. Using a pipette, the contents of the copper/ascorbate vial were transferred to the stirring azide/alkyne vial and capped. The mixture was stirred at room temperature for 1.5-2 hours then diluted with 20 ml of water and added to the top of a 3 kD MWCO spin-filter. The filter was centrifuged at 14,000 rpm until the product was separated. A fresh aliquot of water was added and the washing steps repeated. Washing was repeated for a total of 3 washes.

The retentate was removed using DI water. The retentate formed a sticky solid which only slowly dissolved. A needle was used to dislodge the solids, helping in removal of the retentate. In a falcon tube the retentate was dissolved in DI water using sonication and vortexing. The solution was centrifuged to remove the dissolved material leaving the pellet behind. These steps were repeated two more times to dissolve as much retentate as possible. There was a very small/no pellet remaining after this procedure. Any pellet was discarded and the dissolved material lyophilized. The material was weighed and analyzed for free and bound doxorubicin.

This material showed elevated free doxorubicin so the material was dissolved and washed with pure water using a 3 kD spin filter as discussed herein for a total of 4 extra washes and then lyophilized from pure water.

Table 3 sets forth the compounds and their amounts and characteristics for this example.

TABLE 3

| | MW | Other | | | | |
|---|---|---|---|---|---|---|
| Compound | (g/mol) | Features | Amount | | Moles | Ratio |
| DOX- | 702 | | 50 | mg | 0.00007123 | 12 |
| hydrazone | | | | | | |
| Azide | | | | | | |
| Alkyne | 9137 | 10 alkynes | 54 | mg | 0.00000591 | 1 |
| Copper sulfate | 159.6 | | 5.6 | mg | 0.0000351 | 6 |
| Ligand | 329.4 | | 12 | mg | 0.0000364 | 6 |
| Sodium | 198.11 | | 12 | mg | 0.0000606 | 10 |
| ascorbate | | | | | | |
| DMSO | | | 3 | ml | | 7.5 |
| water | | | 0.4 | ml | | 1 |

This example yielded 62.4 mg (60%). Free doxorubicin was present at 1.19% by mass and bound doxorubicin was at 3 dox/chain (17.52% by mass).

### Example 5

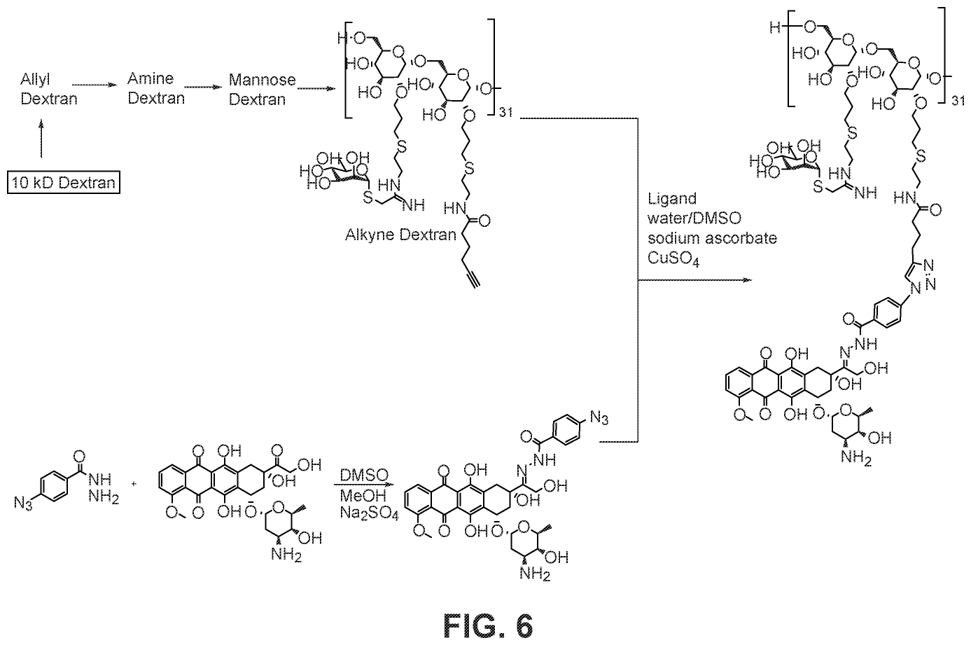

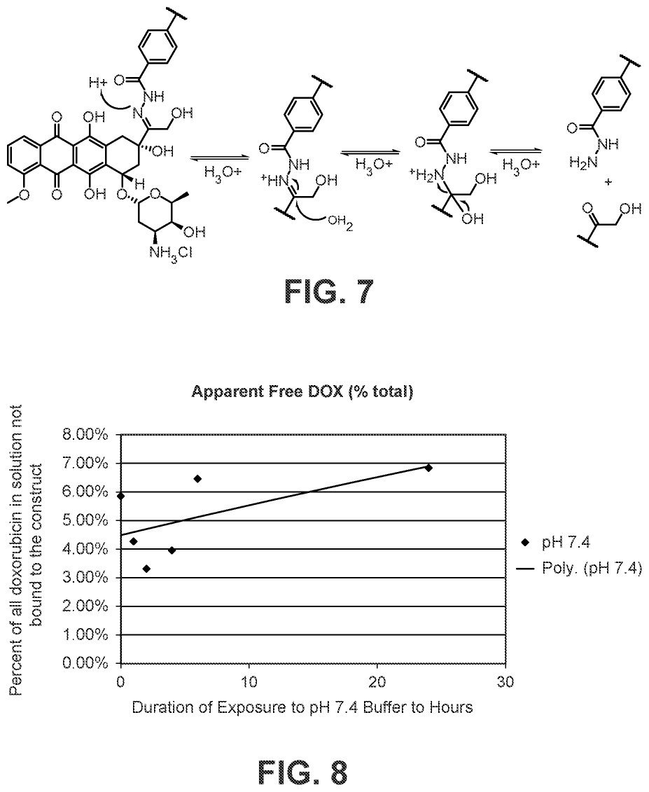

### Synthesis of MAD construct with Doxorubicin Payload Carried on a pH labile Hydrazone linker Using Click Chemistry (FIG. 6 and FIG. 7)

FIG. 6 shows an embodiment of the process used to prepare the doxorubicin construct utilizing a mannose dextran polymer. The example embodiment of FIG. 6 is based on a 10 kDa dextran backbone, but the same procedure is applicable to 3.5 kDa dextran and other carbohydrate polymers of varied Mw ranges as well as disclosed herein. FIG. 7 shows an acid-catalyzed hydrolysis of the hydrazone linker where doxorubicin is released from the final product.

### pH Dependent Release of Free Doxorubicin from the 10 kDa mannose-doxorubicin construct

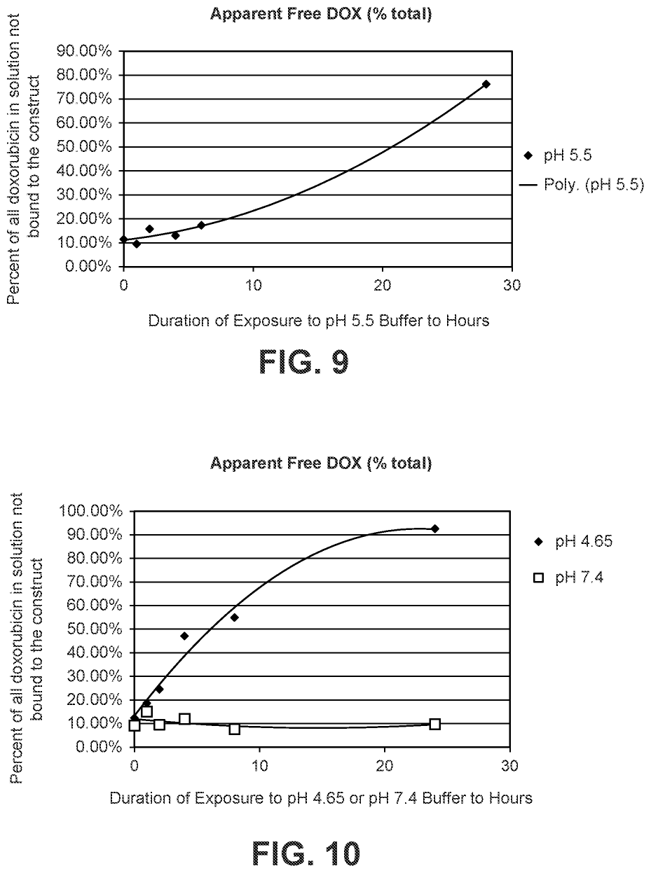

The release of free doxorubicin from the doxorubicin loaded construct over time was profiled in both pH 7.4 buffer (FIG. 8) and pH 5.5 buffer (FIG. 9). A 5 mg/ml solution of the MAN-DOX construct was prepared in both pH 7.4 buffer and pH 5.5 buffer. At the appropriate time points an aliquot of each solution was taken and analyzed by liquid chromatography-mass spectrometry (LCMS). Quantitation was accomplished via comparison of the AUC to a 1 mg/ml standard of pure doxorubicin in water. The results are demonstrated in FIG. 8 and FIG. 9.

### pH Dependent Release of Free Doxorubicin from 3.5 kD mannose-doxorubicin construct

The release of free doxorubicin from the doxorubicin loaded construct over time was profiled in both pH 7.4 and pH 4.65 buffer (FIG. 10). A 5 mg/ml solution of the MAN-DOX construct was prepared in both pH 7.4 buffer and pH 4.65 buffer. At the appropriate time points an aliquot of each solution was taken and analyzed by LCMS. Quantitation was accomplished via comparison of the AUC to a 1 mg/ml standard of pure doxorubicin in water.

FIG. 10 shows minimal release of doxorubicin at pH 7.4 over the course of the experiment. On the other hand, the experiment shows a time dependent release of doxorubicin at pH 4.65 which closely matches a second order polynomial decay. This example shows approximately 80% change in the amount of free doxorubicin over the course of the experiment, with approximately 90% of the doxorubicin being released in 24 hours.

### Example 6

### GPC evaluations of Molecular Weight Distributions of MAN-DOX constructs

Three MAN-DOX constructs were evaluated by gel permeation chromatography (GPC) to determine their molecular weight distributions. One of these three MAN-DOX constructs was a MAN-DOX construct built on a 10 kDa (Mw) dextran backbone using the synthesis pathway described in FIG. 1 (prior art), and a calculated molecular weight (Mw) of approximately 22 kDa. Another of these MAN-DOX constructs was a MAN-DOX construct built on a 10 kDa (Mw) dextran backbone using the CLICK chemistry synthesis pathway disclosed herein and shown in FIG. 6, the formula having a molecular weight (Mw) of approximately 22 kDa. The final MAN-DOX construct was a MAN-DOX construct built on a 3.5 kDa (Mw) dextran backbone using the CLICK chemistry synthesis pathway disclosed herein, the formula having a molecular weight (Mw) of approximately 9 kDa.

The GPC column was equilibrated in a solution of 0.1M LiCl in DMSO and calibrated with a set of Pullulan size standards. For analyses, the three MAN-DOX constructs were dissolved in 0.1M LiCl in DMSO and applied to the GPC column. Samples were eluted from the column in 0.1M LiCl in DMSO and detected by UV absorption at 280 nm and by dynamic measurements of the refractive indices of the eluant. Molecular Weight (Mw) determinations were accomplished by comparing the measured elution volumes to a conventional calibration curve generated by plotting Mw versus elution volume for several Pullman standards. Mw determination was attempted using multi angle light scattering (MALS), however these constructs are not amenable to MALS detection. For each construct, the number-average molecular weight (Mn), the weight-average molecular weight (Mw), and the polydispersity index (PDI), which is Mw/Mn, were determined. For reference, the PDI of the input dextrans were approximately 1.3. Observed increases in Mw compared to calculated formula Mw and in PDI indicate crosslinking and/or oligomerization of the synthesis products. The results of these analyses are shown in Table 3:

TABLE 4

| Construct | Mn | Mw | PDI |
|---|---|---|---|
| 10 kDa backbone with prior art method | 9.9 kDa | 56.4 kDa | 5.69 |
| 10 kDa backbone with CLICK method | 10.5 kDa | 24.6 kDa | 2.35 |
| 3.5 kDa backbone with CLICK method | 5.24 kDa | 8.04 kDa | 1.53 |

The results show that the MAN-DOX construct synthesized by the prior art acid-based synthesis method has an apparent molecular weight (Mw) that is about 2.5 times larger than the calculated formula Mw of approximately 22 kDa, indicating that the material is extensively crosslinked. Conversely, the MAN-DOX construct synthesized using CLICK chemistry on the same sized dextran backbone exhibited a Mw of 24.6 kDa, which is only 12% larger than the calculated formula weight. This result indicates that the 10 kDa backbone construct made by the CLICK method is either not crosslinked or significantly less crosslinked than the MAN-DOX construct synthesized by the prior known method. This difference in crosslinking is also evidenced by the differences in PDI. The observed PDI of the MAN-DOX made by the prior known method is 2.4 times (5.69) larger than the PDI of the MAN-DOX synthesized by the CLICK method (2.35). A MAN-DOX construct built on a 3.5 kDa dextran backbone was also evaluated by the GPC method. The results showed an apparent Mw of 8.04 kDa and a PDI of 1.53, indicating that this construct was also not significantly crosslinked. These results demonstrate that the CLICK method can be applied to MAD constructs of various sizes and is not restricted to constructs built on a 10 kDa dextran backbone.

### Example 7

### Changing the Loading of Doxorubicin

As described herein, one of the advantages of the CLICK synthesis method over the prior art is the ability to reliably and predictably control the loading of doxorubicin onto the mannose-dextran backbone. This is accomplished because of the precise nature of the click chemistry. Accordingly, varying the ratio of the mannose dextran bound alkyne to the doxorubicin bound azide has been found to change the overall loading of doxorubicin in the final product. Examples of how varying the ratios of alkyne to DOX-hydrazone azide can alter the overall loading of doxorubicin are shown in Table 4 (see Formula 1 versus Formula 2 and Formula 4 versus Formula 5). Changing the catalyst loadings alone had minimal effect on the doxorubicin loading in the final product (see Formula 2 versus Formula 5 and Formula 3 versus Formula 4).

TABLE 5
Control of loading ratio as a function of azide to alkyne ratio

| Formula | 1 | 2 | 3 | 4 | 5 |
|---|---|---|---|---|---|
| Moles polymer | 1 | 1 | 1 | 1 | 1 |
| Moles alkyne | 12 | 21.7 | 21.7 | 21.7 | 21.7 |
| Moles DOX- | 12 | 12 | 22 | 22 | 12 |
| hydrazone azide | | | | | |
| Moles catalyst | 6 | 6 | 11 | 6 | 11 |
| (i.e., Cu and ligand) | | | | | |
| Dox/chain | 6.7 | 3.2 | 9.2 | 7.3 | 3.5 |

By comparing the entries in Table 4 above, the following observations can be made regarding reaction conditions. In certain embodiments, the ratio of alkyne to catalyst that obtains the most desirable results is about 2:1. With higher alkyne to azide ratios, the loading of doxorubicin goes down (see Formula 1 versus Formula 2). This trend can be somewhat reversed by adding more doxorubicin (see Formula 2 versus Formula 4). In addition to these conditions, a ligand to catalyst ratio of 1:1, an ascorbate to catalyst ratio of about 5:3, and a water to DMSO ratio of about 1:7.5 were found to create desirable results.

Although the disclosure has been described with references to various embodiments, persons skilled in the art will recognized that changes may be made in form and detail without departing from the spirit and scope of this disclosure.

What is claimed is:

1. A method for conjugating anthracycline to a carbohydrate polymer comprising:
providing (1) a carbohydrate polymer comprising at least one alkyne moiety bound directly or indirectly thereto, and (2) an anthracycline derivatized to a hydrazone azide; or
providing (1) a carbohydrate polymer comprising at least one hydrazone azide moiety bound directly or indirectly thereto, and (2) an anthracycline comprising at least one alkyne moiety bound directly or indirectly thereto; and
reacting the carbohydrate polymer with the anthracycline in the presence of a Cu¹ catalyst and an amine ligand to form a 1,3-triazole linkage between the anthracycline and the carbohydrate polymer,
wherein the 1,3-triazole linkage results from a 1,3-cycloaddition between the azide and the alkyne.

2. The method of claim 1, wherein the carbohydrate polymer is selected from the group consisting of cellulose, dextran, and mannan.

3. The method of claim 1, wherein the anthracycline comprises doxorubicin.

4. A method of synthesizing an anthracycline carbohydrate polymer construct comprising:
providing an alkyne carbohydrate polymer;
providing an anthracycline hydrazone azide; and
reacting the alkyne carbohydrate polymer with the anthracycline hydrazone azide in the presence of a Cu¹ catalyst and a ligand to form a 1,3-triazole linkage between the anthracycline and carbohydrate polymer,
wherein the 1,3-triazole linkage results from a 1,3-cycloaddition between the azide of the anthracycline hydrazone azide and the alkyne of the alkyne carbohydrate polymer.

5. The method of claim 4, wherein the anthracycline hydrazone azide is formed by a step comprising derivatizing the anthracycline by reacting the anthracycline with a hydrazide via condensation to form the anthracycline hydrazone azide.

6. The method of claim 4, wherein the hydrazide is 4-azidobenzohydrazide.

7. The method of claim 4, wherein the anthracycline comprises doxorubicin.

8. The method of claim 4, wherein the alkyne carbohydrate polymer construct is synthesized by a step comprising reacting an amine carbohydrate polymer comprising cellulose, dextran, or mannan, with an alkyne under dehydrative conditions to form the alkyne carbohydrate polymer.

9. The method of claim 8, wherein the alkyne is hexynoic acid.

10. The method of claim 4, wherein the ligand is an amine ligand.

11. The method of claim 10, wherein the amine ligand has the following structure:

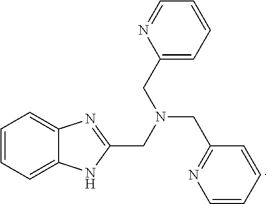 [SMILES: c1ccc(CN(Cc2ccccn2)Cc2nc3ccccc3[nH]2)nc1]

12. The method of claim 11, wherein the ligand is synthesized through the reaction of ((2-benzimidazolyl)methyl)amine and 2-(chloromethyl)pyridine in the presence of Et₃N.

13. The method of claim 4, wherein the ligand and Cu¹ catalyst are present at a ratio of between about 1:5 to about 5:1.

14. The method of claim 4, wherein the step of reacting the alkyne carbohydrate polymer with the anthracycline hydrazone azide is carried out in the presence of sodium ascorbate.

15. The method of claim 14, wherein the Cu¹ catalyst and ascorbate are present at a ratio of between about 1:5 to about 9:10.

16. The method of claim 4, wherein the alkyne and the Cu¹ catalyst are present at a ratio of between about 1:1 to about 5:1.

17. The method of claim 4, wherein the reacting step is performed in the presence of DMSO and water, and wherein the DMSO and water are present at a ratio of between about 4:1 to about 20:1.

18. The method of claim 4, wherein loading of anthracycline to the anthracycline carbohydrate polymer construct can be controlled by varying the ratio of alkyne to the anthracycline hydrazone azide.

19. The method of claim 18, wherein as the ratio of the alkyne to anthracycline hydrazone azide increases, the loading of anthracycline to the anthracycline carbohydrate polymer construct decreases.

20. The method of claim 4, wherein the alkyne carbohydrate polymer is a mannosylated alkyne carbohydrate polymer having a molecular weight (Mw) of from about 1 kDa to about 50 kDa.

21. The method of claim 4, wherein the alkyne carbohydrate polymer has the structure of formula (I): 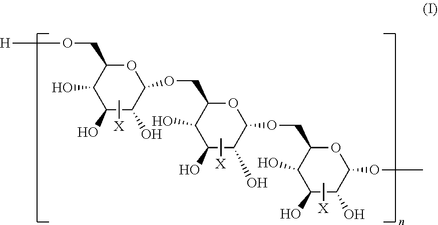
wherein
each X is independently H, L2-R, or L3-Y;
each L2 and L3 are independently amino terminated leashes comprising the formula (CH₂)ₚS(CH₂)_{q}—NH—, wherein p and q are integers from 0 to 5;
each R independently comprises a mannose-binding C-type lectin receptor targeting moiety, or H;
each Y independently comprises a terminal alkyne moiety, or H; and
n is an integer greater than zero,
wherein at least one R comprises a mannose-binding C-type lectin receptor targeting moiety selected from the group consisting of mannose, fucose, and n-acetylglucosamine;
wherein at least one Y comprises the terminal alkyne moiety; and
wherein each unit of n may be the same or different.

22. The method of claim 21, wherein each X is independently H, 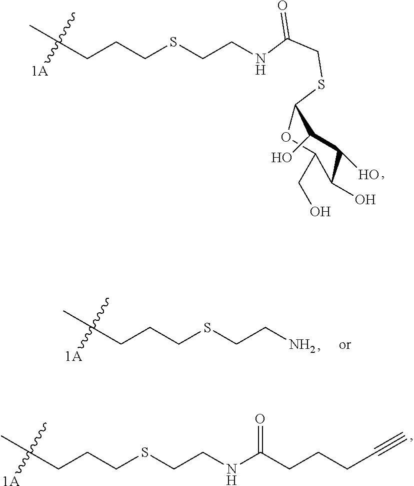
and wherein each bond 1A is connected to any —OH group in formula (I).

23. The method of claim 4, wherein the reacting step is carried out at a pH of from about 6.5 to about 10.

24. The method of claim 4, wherein the anthracycline is released from the anthracycline carbohydrate polymer construct at a pH of about 5.5 or below.

25. The method of claim 4, wherein the anthracycline carbohydrate polymer has the structure of formula (II): 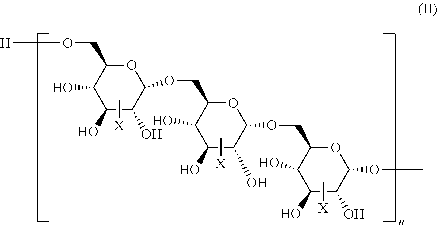
wherein
each X is independently H, L1-A, L2-R, or L3-Y;
each L1, L2 and L3 are independently amino terminated leashes comprising the formula —(CH₂)ₚS(CH₂)_{q}—NH—, wherein p and q are integers from 0 to 5;
each A independently comprises an anthracycline hydrazone azide moiety, or H;
each R independently comprises a mannose-binding C-type lectin receptor targeting moiety, or H;
each Y independently comprises a terminal alkyne moiety, or H; and
n is an integer greater than zero,
wherein at least one A comprises the anthracycline hydrazone azide moiety;
wherein at least one R comprises a mannose-binding C-type lectin receptor targeting moiety selected from the group consisting of mannose, fucose, and n-acetylglucosamine;
wherein at least one Y comprises the terminal alkyne moiety; and
wherein each unit of n may be the same or different.

26. The method of claim 25, wherein each X is independently H, 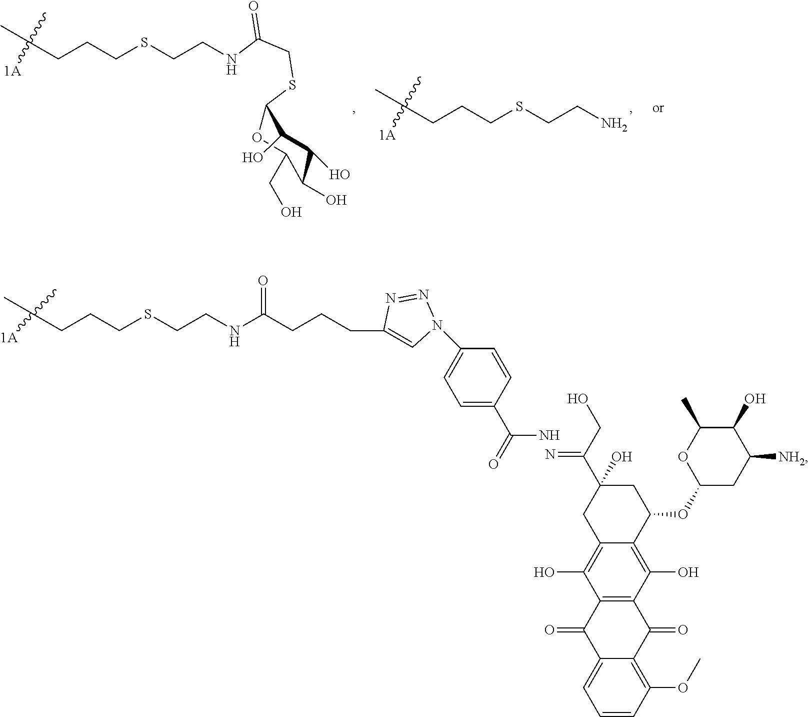
and wherein each bond 1A is connected to any —OH group in formula (II).

27. A pharmaceutical composition comprising:
a compound having the structure of formula (II): 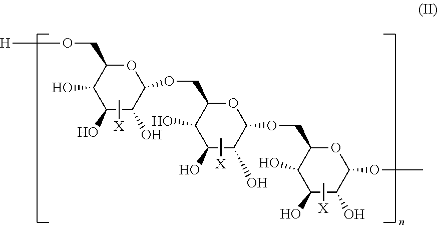
wherein
each X is independently H, L1-A, L2-R, or L3-Y;
each L1, L2 and L3 are independently amino terminated leashes comprising the formula —(CH₂)ₚS(CH₂)_{q}—NH—, wherein p and q are integers from 0 to 5;
each A independently comprises an anthracycline hydrazone azide moiety, or H;
each R independently comprises a mannose-binding C-type lectin receptor targeting moiety, or H;
each Y independently comprises a terminal alkyne moiety, or H; and
n is an integer greater than zero,
wherein at least one A comprises the anthracycline hydrazone azide moiety;
wherein at least one R comprises a mannose-binding C-type lectin receptor targeting moiety selected from the group consisting of mannose, fucose, and n-acetylglucosamine;
wherein at least one Y comprises the terminal alkyne moiety;
wherein each unit of n may be the same or different; and
a pharmaceutically acceptable carrier thereof.

28. The pharmaceutical composition of claim 27, wherein each X is independently is H, 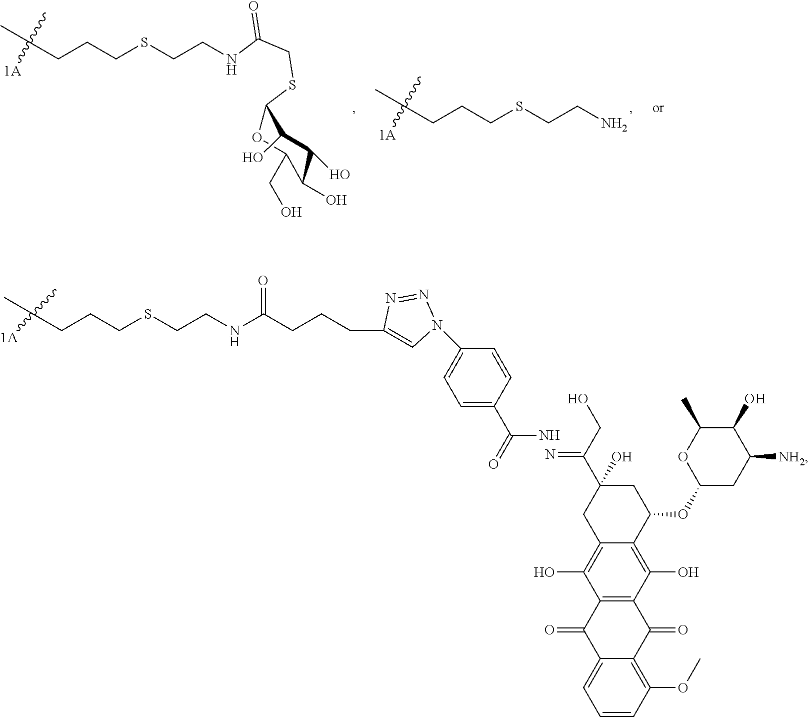
and wherein each bond 1A is connected to any —OH group in formula (II).

29. The pharmaceutical composition of claim 27, wherein the compound of formula (II) has a molecular weight (Mw) of greater than about 5 kDa.

30. The pharmaceutical composition of claim 27, wherein the anthracycline comprises doxorubicin.

31. The pharmaceutical composition of claim 27, wherein the anthracycline is released from the composition at a pH of about 5.5 or below.
